(19) United States
(12) Patent Application Publication
(10) Pub. No.: US 2020/0337576 A1
(43) Pub. Date: Oct. 29, 2020

(54) INSPECTION APPARATUS, METHOD OF OPERATING INSPECTION APPARATUS, AND COMPUTER-READABLE MEDIUM

(71) Applicant: NIHON KOHDEN CORPORATION, Tokyo (JP)

(72) Inventors: Hiroyuki MATSUMOTO, Tokorozawa-shi (JP); Natsuki FUJIWARA, Tokorozawa-shi (JP); Akira IZUMIDA, Tokorozawa-shi (JP); Takuya ISHIGURO, Tokorozawa-shi (JP)

(21) Appl. No.: 16/850,683

(22) Filed: Apr. 16, 2020

(30) Foreign Application Priority Data
Apr. 25, 2019 (JP) 2019-084326

(51) Int. Cl. A61B 5/0402; G06F 3/0488

(52) U.S. Cl. CPC A61B 5/0402; G06F 3/0488

(57) ABSTRACT

An inspection apparatus to inspect physiological information of a subject being tested includes: an input interface configured to receive a signal corresponding to the physiological information; a display configured to display information corresponding to the signal in real time during the inspection; a storage in which data corresponding to the information is stored; a communication interface communicatively connected to an external apparatus; and one or more processor configured to, when a request from the external apparatus is received by the communication interface, generate based on the data a data file for enabling browsing of the information and transmit the data file to the external apparatus via the communication interface.

### CROSS-REFERENCE TO RELATED APPLICATIONS

This application is based on and claims priority under 35 USC 119 from Japanese Patent Application No. 2019-084326 filed on Apr. 25, 2019, the contents of which are incorporated herein by reference.

### TECHNICAL FIELD

The presently disclosed subject matter relates to an inspection apparatus that is to be used for inspection of physiological information of a subject being tested. The presently disclosed subject matter also relates to a method of operating the inspection apparatus and a computer-readable medium storing a program for controlling the inspection apparatus.

### BACKGROUND

Japanese Patent Application Laid-Open Publication No. 2018-171239 discloses an electrocardiograph, as an example of the inspection apparatus. The electrocardiograph is configured to acquire signals corresponding to an electrocardiogram of the subject being tested, through a plurality of electrodes attached to the subject being tested. The electrocardiograph includes a display unit. An electrocardiographic waveform corresponding to signals acquired during inspection is displayed in real time on the display unit. Data corresponding to the electrocardiographic waveform is stored in a storage such as a memory card, as an inspection result.

An object of the presently disclosed subject matter is to improve convenience relating to management of data stored as an inspection result.

### SUMMARY

An inspection apparatus to inspect physiological information of a subject being tested includes: an input interface configured to receive a signal corresponding to the physiological information; a display configured to display information corresponding to the signal in real time during the inspection; a storage in which data corresponding to the information is stored; a communication interface communicatively connected to an external apparatus; and one or more processor configured to, when a request from the external apparatus is received by the communication interface, generate based on the data a data file for enabling browsing of the information and transmit the data file to the external apparatus via the communication interface.

### BRIEF DESCRIPTION OF DRAWINGS

Exemplary embodiments of the present invention will be described in detail based on the following figures, wherein:

FIG. 1 illustrates an example of a configuration of an electrocardiogram management system in accordance with an embodiment;

FIG. 2 illustrates an example of a configuration of an electrocardiograph configuring the electrocardiogram management system;

FIG. 3 illustrates an example of an operation of one or more processor of the electrocardiograph;

FIG. 4 illustrates an example of an image that is to be browsed on an external apparatus;

FIG. 5A illustrates an example of an image that is to be browsed on the external apparatus;

FIG. 5B illustrates an example of an image that is to be browsed on the external apparatus; and

FIG. 6 illustrates an example of an image that is to be browsed on the external apparatus.

### DESCRIPTION OF EMBODIMENTS

Hereinbelow, an embodiment will be described in detail with reference to the accompanying drawings.

In the respective drawings, a scale is appropriately changed so that each element to be described can be recognized.

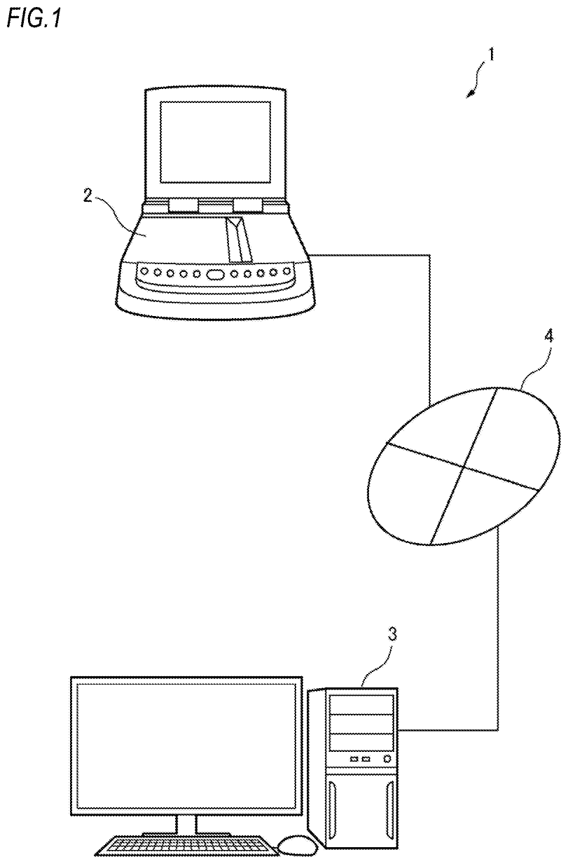

FIG. 1 illustrates an example of a configuration of an electrocardiogram management system 1 in accordance with an embodiment. The electrocardiogram management system 1 can include an electrocardiograph 2 and an external apparatus 3. The electrocardiograph 2 and the external apparatus 3 are communicatively connected to each other via a communication network 4. The communication network 4 may include at least one of a local area network (LAN) established in a healthcare center and a WAN such as Internet.

The electrocardiograph 2 is an example of an inspection apparatus that is to be used for electrocardiogram inspection of a subject being tested. The external apparatus 3 is an apparatus capable of displaying information on a display apparatus, in addition to communication with the electrocardiograph 2. The display apparatus may be provided as a part of the external apparatus 3 or as a separate apparatus from the external apparatus 3.

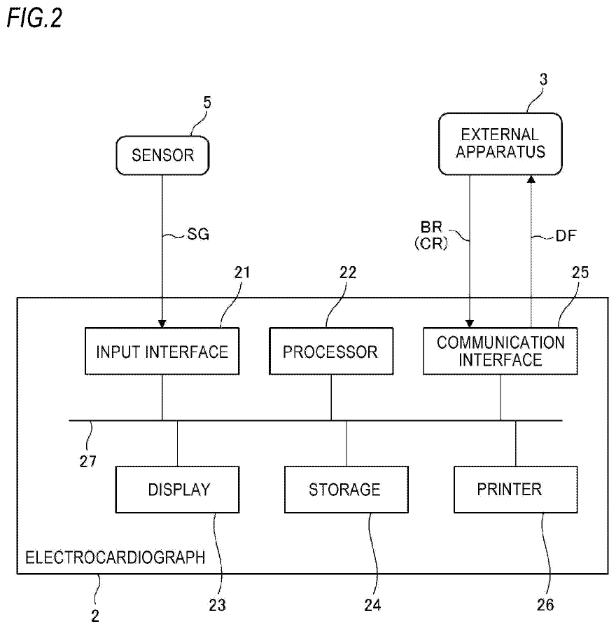

FIG. 2 exemplifies a functional configuration of the electrocardiograph 2. The electrocardiograph 2 is configured to acquire a signal SG corresponding to an electrocardiogram of a subject being tested, through a sensor 5 mounted on the subject being tested. Specifically, the sensor 5 can include a plurality of electrodes to be mounted on a plurality of body parts of the subject being tested. Therefore, the signal SG includes a plurality of signals corresponding to potentials of the body parts to which the respective electrodes are mounted.

The electrocardiograph 2 can include an input interface 21 and one or more processor 22. The input interface 21 is configured to receive the signal SG output from the sensor 5. The one or more processor 22 is configured to execute a variety of processing to be described later, based on the signal SG received by the input interface 21. The input interface 21 can includes an appropriate signal processing circuit configured to convert the signal SG into a signal that can be processed by the one or more processor 22, as required.

The electrocardiograph 2 can include a display 23. The display 23 is configured to display an electrocardiographic waveform corresponding to the signal SG in real time during inspection. The one or more processor 22 is configured to display change over time in potential of the signal SG on the display 23, as an electrocardiographic waveform. The electrocardiographic waveform is an example of information corresponding to a signal.

The electrocardiograph 2 can include a storage 24. The one or more processor 22 is configured to generate data corresponding to the information displayed on the display 23, and to store the same in the storage 24, as an inspection result. The storage 24 may be implemented by a hard disk device or a semiconductor memory. The storage 24 may be detachably mounted to the electrocardiograph 2.

The data is stored in the storage 24, as a unit of one inspection. That is, data corresponding to a result of at least one inspection performed for at least one subject being tested is stored in the storage 24.

The electrocardiograph 2 can include a communication interface 25. The communication interface 25 is configured to perform communication with the external apparatus 3 via the communication network 4. The communication with the external apparatus may be performed in a wired manner or in a wireless manner.

The electrocardiograph 2 can include a printer 26. The printer 26 is configured to print on a recording sheet the electrocardiographic waveform displayed on the display 23. An operation of the printer 26 is controlled by the one or more processor 22.

The electrocardiograph 2 can include a communication bus 27. The communication bus 27 is configured to enable communication of signal and data among the input interface 21, the one or more processor 22, the display 23, the storage 24, the communication interface 25, and the printer 26.

The external apparatus 3 is configured to activate a browser as software for enabling data to be browsed on the display apparatus. A browsing target by the browser is a specific electrocardiogram inspection result. A user of the external apparatus 3 can request the electrocardiograph 2 for browsing of an inspection result. For example, an application configured to operate on the browser is activated, so that a browsing request BR can be output from the external apparatus 3 to the communication network 4. The browsing request BR is received by the communication interface 25 of the electrocardiograph 2.

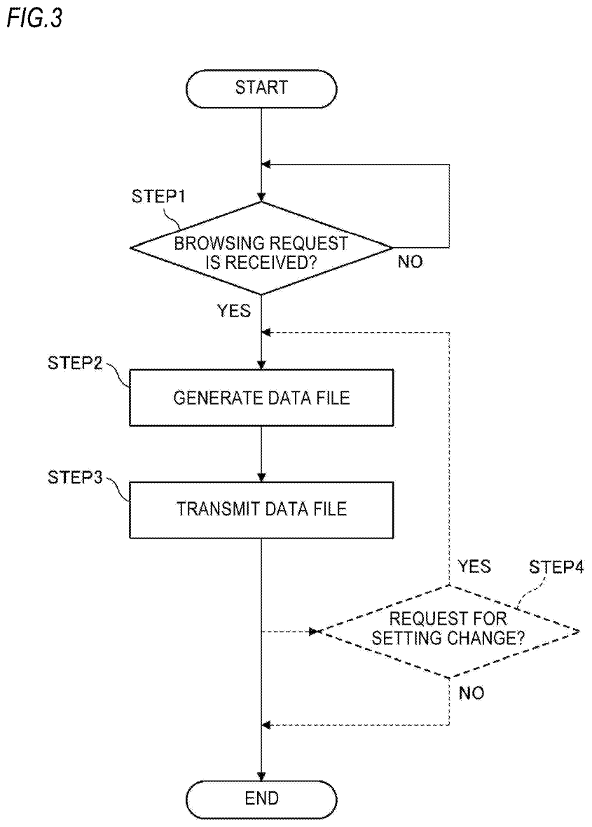

FIG. 3 exemplifies processing that is to be executed by the one or more processor 22. The processing is executed independently of the real time display of the electrocardiographic waveform on the display 23. In other words, even while the processing is executed by the one or more processor 22, the electrocardiogram inspection can proceed.

The one or more processor 22 determines whether the communication interface 25 has received a browsing request BR (STEP1). Until it is determined that the browsing request BR is received, the processing is repeated (NO in STEP1).

When it is determined that the communication interface 25 has received a browsing request BR (YES in STEP1), the one or more processor 22 generates a data file DF for enabling browsing of the information corresponding to the browsing request BR, based on the data stored in the storage 24 (STEP2).

The data file DF may include a list of inspection results, for example. The list of inspection results may include, as information, date and time at which inspection was performed, an identification number of a subject being tested, a name of the subject being tested, a sex of the subject being tested, and the like. The one or more processor 22 reads data corresponding to the information from the storage 24, and makes the data file DF for enabling browsing of the information.

Continuously, the one or more processor 22 transmits the generated data file DF to the external apparatus 3 via the communication interface 25 (STEP3).

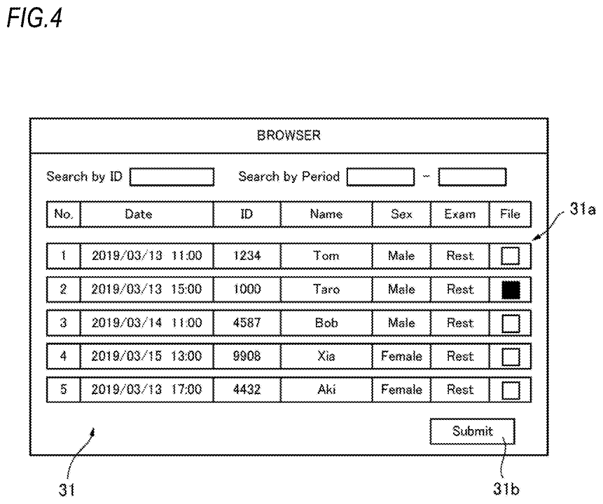

The external apparatus 3 causes the browser to display an image 31 indicative of the list of inspection results, based on the received data file DF. FIG. 4 illustrates an example of the image 31. As described above, the list may include, as the information, date and time at which inspection was performed, an identification number of a subject being tested, a name of the subject being tested, a sex of the subject being tested, and the like. Also, the image 31 may include check boxes 31a for selection, and a transmission button image 31b. The user of the external apparatus 3 can select at least one inspection that the user wants to browse. In the shown example, only the inspection “No. 2” is selected. When the selection is completed, the user clicks or touches the transmission button image 31b.

By the above operation, the browsing request BR to request browsing of the selected inspection result is output from the external apparatus 3. In the similar manner to the above, the browsing request BR is received by the communication interface 25 of the electrocardiograph 2 (STEP1 in FIG. 3). The one or more processor 22 generates a data file DF for enabling browsing of the selected inspection result, based on the data stored in the storage 24 (STEP2 in FIG. 3).

The selected inspection result may include an image of an electrocardiographic waveform previously displayed on the display 23, for example. The one or more processor 22 reads data corresponding to the image from the storage 24, and makes a data file DF for enabling browsing of the image. The one or more processor 22 transmits the generated data file DF to the external apparatus 3 via the communication interface 25 (STEP3 in FIG. 3).

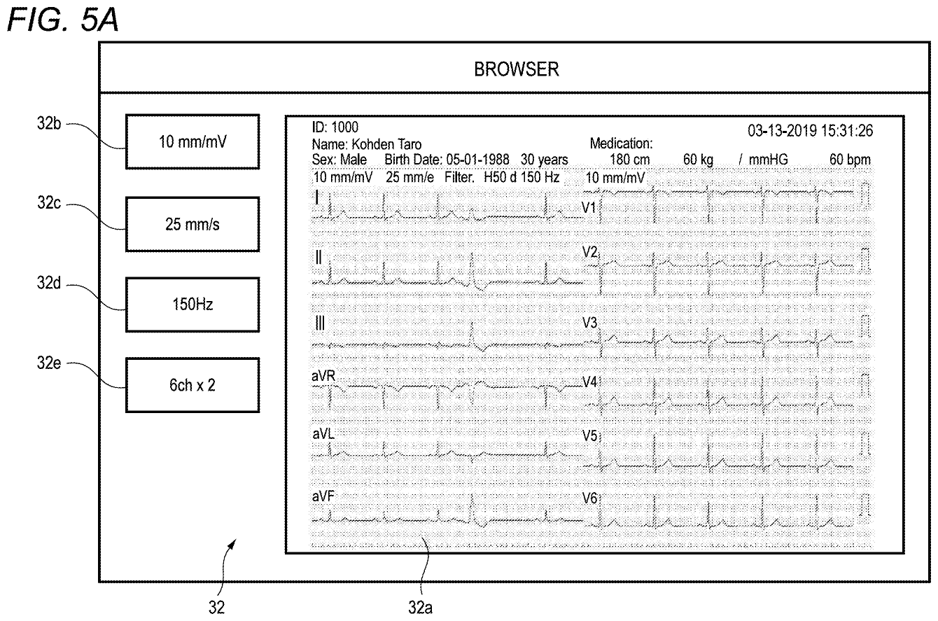

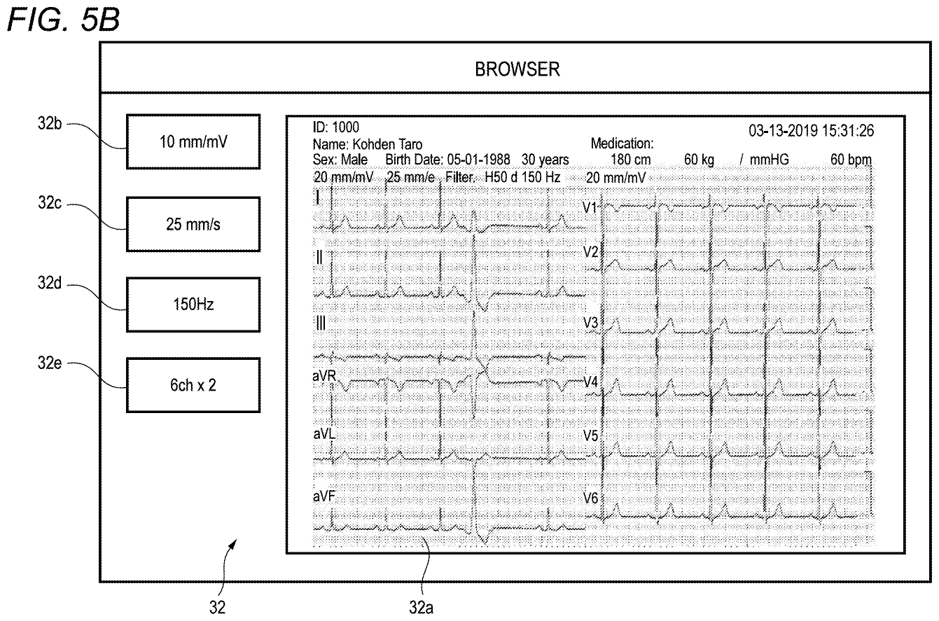

The external apparatus 3 causes the browser to display an image including the selected inspection result, based on the received data file DF. FIG. 5A illustrates an example of an image 32. The image 32 includes an inspection result image 32a. The inspection result image 32a corresponds to electrocardiographic waveforms recorded on the recording sheet by the conventional method.

As described above, a series of processing of generating the data file DF for enabling browsing of the information previously displayed on the display 23, based on the data stored in the storage 24, and transmitting the data file DF to the external apparatus 3 are performed independently of the real time display of the electrocardiographic waveform on the display 23. That is, the electrocardiograph 2 can be enabled to function as a sever for the external apparatus 3 as a client terminal. Therefore, even in a situation in which the electrocardiograph 2 is used for electrocardiogram inspection of any subject being tested, the external apparatus 3 can browse other inspection performed for the subject being tested or a result of inspection performed for another subject being tested. Accordingly, it is possible to improve convenience relating to management of data stored as an inspection result.

Particularly, in the case of the electrocardiograph 2, an inspection result is generally recorded on a recording sheet. In particular, in a case in which a heat sensitive sheet is used as the recording sheet, care is required for long-term storage management because the sheet is likely to be deteriorated due to heat. According to the configuration as described above, since it is possible to browse the inspection result, as required, without depending on the recording on the sheet, it is possible to reduce burden on data management. Also, the printing on the recording sheet has only to be performed when necessary, which contributes to saving sheet resources. The printer 26 may be omitted if the data can be browsed only on the external apparatus 3.

As shown in FIG. 5A, the image 32 may include a plurality of button images 32b to 32e for changing a variety of display settings, in addition to the inspection result image 32a.

The button image 32b is operated so as to change a display sensitivity. The “display sensitivity” is a scale corresponding to a length of the recording sheet in a vertical axis direction per unit voltage. For example, when the button image 32b is clicked or touched, the display sensitivity is switched between 10 mm/mV and 20 mm/mV.

The button image 32c is operated so as to change a display speed. The “display speed” is a scale corresponding to a moving speed of the recording sheet. In the inspection result image 32a, the display speed corresponds to a length in a horizontal axis direction per unit time. For example, when the button image 32b is clicked or touched, the display speed is switched between 25 mm/second and 50 mm/second.

The button image 32d is operated so as to change a display filter. The “display filter” is used so as to remove noises mixed into the signal SG. For example, when the button image 32b is clicked or touched, a filtering frequency of the display filter is switched among 25 Hz, 35 Hz, 75 Hz, 100 Hz, and 150 Hz, for example.

The button image 32e is operated so as to change a display channel. The “display channel” is used so as to designate the numbers of induction waveforms, which configure the electrocardiogram, aligned in the vertical direction and in the horizontal direction, respectively. For example, in a case in which induction waveforms of 12 channels are displayed, when the button image 32b is clicked or touched, the display channel is switched among a mode in which induction waveforms of 3 channels are aligned and displayed in 4 rows, a mode in which induction waveforms of 6 channels are aligned and displayed in 2 rows, and a mode in which induction waveforms of 12 channels are aligned and displayed in one row, for example.

When any one of the button images 32b to 32e is operated, the external apparatus 3 may output a change request CR for changing a browsing mode, as shown in FIG. 2. In this case, as shown in FIG. 3, the one or more processor 22 may determine whether the communication interface 25 has received the change request CR after transmitting the data file DF (STEP4). When it is not determined that the change request CR has been received (NO in STEP4), the processing is over.

When it is determined that the change request CR has been received by the communication interface 25 (YES in STEP4), the one or more processor 22 generates a data file DF for enabling browsing corresponding to the change request CR, based on the data stored in the storage 24 (STEP2). That is, the one or more processor 22 changes the configuration of the data file DF to be transmitted, in response to the change request CR.

Continuously, the one or more processor 22 transmits the generated data file DF to the external apparatus 3 via the communication interface 25 (STEP3).

For example, in the image 32 of FIG. 5A, the display sensitivity is set to 10 mm/mV. When the button image 32b is operated to change the display sensitivity to 20 mm/mV, the inspection result image 32a is changed, as shown in FIG. 5B.

According to the above configuration, a display mode of the inspection result is changed in an interactive manner from the external apparatus 3, so that an optimal browsing environment can be obtained. Accordingly, it is possible to improve convenience relating to management of data stored as an inspection result.

Particularly, in a case in which an inspection result is printed on a recording sheet, the re-printing is required so as to change a display mode. However, as described above, the interactive change of the display mode is allowed, so that it is possible to minimize the printing on the recording sheet.

The one or more processor 22 can provide an analysis result of the information displayed on the display 23, based on the data stored in the storage 24. As the information to be provided, opinions on the acquired electrocardiographic waveform (sinus rhythm, arrhythmia, tachycardia, bradycardia, premature ventricular contraction, and the like), representative measurement values, a waveform (average waveform) obtained by performing time averaging processing on each induction waveform, a waveform (rhythm waveform) in which collected waveforms over entire time are displayed by designated induction, and the like may be exemplified.

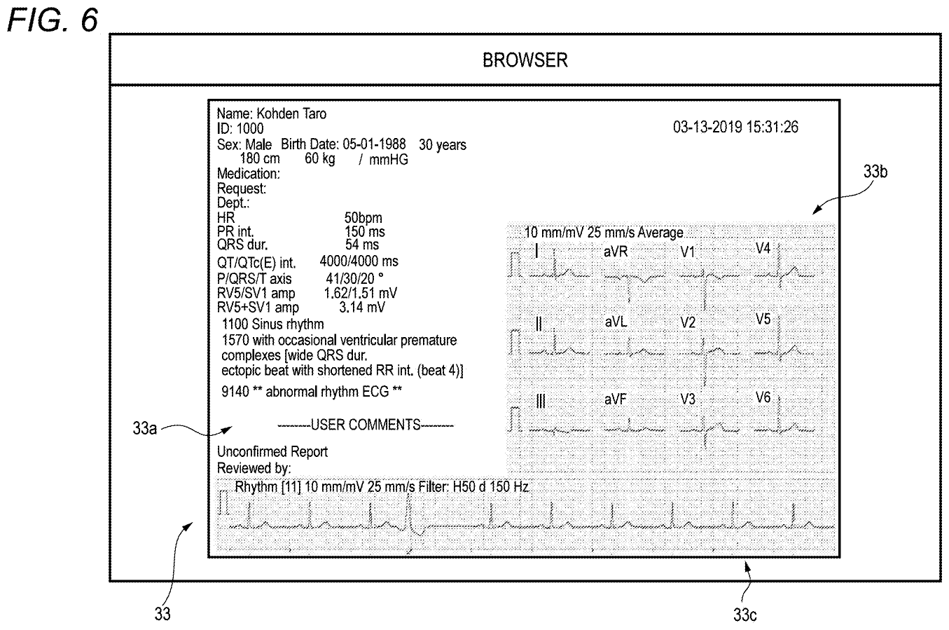

In addition to or alternatively to the inspection results shown in FIGS. 5A and 5B, the one or more processor 22 may generate a data file DF so as to include the analysis result. FIG. 6 illustrates an example of an image 33 displayed on the browser based on the data file DF including the analysis result. The image 33 includes areas 33a to 33c. The area 33a includes, as information, opinions and representative measurement values. The area 33b includes, as information, average waveforms. The area 33c includes, as information, a rhythm waveform. That is, the electrocardiographic waveform included in the analysis result may be different from the electrocardiographic waveform (the waveform displayed in real time on the display 23) included in the inspection result exemplified in FIGS. 5A and 5B.

The electrocardiograph 2 and the external apparatus 3 are preferably configured to operate on a Web system. In this case, the browser configured to operate on the external apparatus 3 may be a general-purpose Web browser. The electrocardiograph 2 may function as a Web server for the external apparatus as a client terminal. According to this configuration, a general-purpose terminal apparatus can be used as the external apparatus 3. Since it is not necessary to use a dedicated terminal device or dedicated browsing software for browsing of the inspection result, it is possible to improve convenience relating to management of data stored as an inspection result.

The data file DF generated by the one or more processor 22 is preferably based on a PDF format having high compatibility between the software. Even with the configuration, it is possible to easily use a general-purpose terminal device, as the external apparatus 3.

The one or more processors 22 having the above-described functions may be implemented by one or more general-purpose microprocessors configured to operate in cooperation with one or more general-purpose memory. As the general-purpose microprocessor, a CPU and an MPU may be exemplified. As the general-purpose memory, a RAM and a ROM may be exemplified. In this case, a computer program for causing the one or more processors 22 to perform the above-described processing may be stored in the ROM. The one or more processors 22 designate at least a part of the computer program stored on the ROM and develops the same on the RAM, thereby executing the above-described processing in cooperation with the RAM. A part of the storage 24 may also be used as the general-purpose memory. The one or more processors 22 may be implemented by a dedicated integrated circuit such as a microcontroller, an ASIC, an FPGA and the like capable of executing the computer program configured to implement the above-described processing. The one or more processors 22 may also be implemented by a combination of one or more general-purpose microprocessors and a dedicated integrated circuit.

As described above, a part of the general-purpose memory or storage 24 configured to cooperate with the one or more general-purpose microprocessor may be a storage medium in which a computer program configured to control operations of the electrocardiograph 2 is stored. However, the electrocardiograph 2 can be configured to perform communication with an external server (not shown) via the communication network 4. In this case, the computer program can be downloaded from the external server via the communication network 4. The external server may be a storage medium in which the computer program configured to control operations of the electrocardiograph 2 is stored.

The embodiment is just exemplary so as to easily understand the presently disclosed subject matter. The configuration of the embodiment can be appropriately changed and improved without departing from the gist of the presently disclosed subject matter.

As the inspection apparatus by which the information displayed on the display 23 is printed on the recording sheet, an electroencephalograph may be exemplified, in addition to the electrocardiograph. Therefore, when the above-described configuration is applied to the electroencephalograph, the advantageous effects can also be realized. However, the above-described configuration can also be applied to an inspection apparatus that does not involve the printing on the recording sheet.

The aforementioned embodiments are summarized as follows.

As a first aspect of the presently disclosed subject matter, an inspection apparatus to inspect physiological information of a subject being tested includes: an input interface configured to receive a signal corresponding to the physiological information; a display configured to display information corresponding to the signal in real time during the inspection; a storage in which data corresponding to the information is stored; a communication interface communicatively connected to an external apparatus; and one or more processor configured to, when a request from the external apparatus is received by the communication interface, generate based on the data a data file for enabling browsing of the information and transmit the data file to the external apparatus via the communication interface.

A series of processing of generating the data file for enabling browsing of the information previously displayed on the display, based on the data stored in the storage, and transmitting the data file to the external apparatus are performed independently of the real time display of the electrocardiographic waveform on the display. That is, the electrocardiograph can function as a server for the external apparatus as a client terminal. Therefore, even in a situation in which the electrocardiograph is used for electrocardiogram inspection of any subject being tested, the external apparatus can browse other inspection performed for the subject being tested or a result of inspection performed for another subject being tested. Accordingly, it is possible to improve convenience relating to management of data stored as an inspection result.

As used herein, the expression “display in real time” means that change over time in condition of the subject being tested during inspection is reflected in change over time in display on the display unit. The expression may include a case in which delay occurs with respect to signal processing and display processing after the physiological information of the subject being tested is acquired before the information is displayed on the display unit.

As a second aspect of the presently disclosed subject matter, a method of operating an inspection apparatus to inspect physiological information of a subject being tested includes: receiving a signal corresponding to the physiological information; displaying information corresponding to the signal in real time on a display during the inspection; storing data corresponding to the information; generating based on the data a data file for enabling browsing of the information in response to a request from an external apparatus; and transmitting the data file to the external apparatus.

As a third aspect of the presently disclosed subject matter, a non-transitory computer-readable medium stores a program for causing an inspection apparatus to inspect physiological information of a subject being tested to execute a process. The process includes: receiving a signal corresponding to the physiological information; displaying information corresponding to the signal in real time on a display during the inspection; storing data corresponding to the information; generating based on the data a data file for enabling browsing of the information in response to a request from an external apparatus; and transmitting the data file to the external apparatus.

What is claimed is:

1. An inspection apparatus to inspect physiological information of a subject being tested, the inspection apparatus comprising:
an input interface configured to receive a signal corresponding to the physiological information;
a display configured to display information corresponding to the signal in real time during the inspection;
a storage in which data corresponding to the information is stored;
a communication interface communicatively connected to an external apparatus; and
one or more processor configured to, when a request from the external apparatus is received by the communication interface, generate based on the data a data file for enabling browsing of the information and transmit the data file to the external apparatus via the communication interface.

2. The inspection apparatus according to claim 1, wherein the one or more processor is configured to change a configuration of the data file to be transmitted, in response to a request to change a browsing mode from the external apparatus.

3. The inspection apparatus according to claim 1, wherein the one or more processor is configured to provide an analysis result of the information, as a part of the data file.

4. The inspection apparatus according to claim 1, wherein the data file is based on a PDF format.

5. The inspection apparatus according to claim 1, wherein
the physiological information is an electrocardiogram or electroencephalogram, and
a printer configured to print information displayed on the display is provided.

6. The inspection apparatus according to claim 1, wherein
the display is further configured to display a waveform in real time, and
the one or more processor is further configured to generate a data file based on data corresponding to information previously displayed on the display.

7. The inspection apparatus according to claim 1, wherein
the one or more processor is further configured to generate a data file of another inspection or an inspection on another day that has been performed for a certain subject being tested, in a state in which an inspection is being performed for the certain subject being tested.

8. A method of operating an inspection apparatus to inspect physiological information of a subject being tested, the method comprising:
receiving a signal corresponding to the physiological information;
displaying information corresponding to the signal in real time on a display during the inspection;
storing data corresponding to the information;
generating based on the data a data file for enabling browsing of the information in response to a request from an external apparatus; and
transmitting the data file to the external apparatus.

9. A non-transitory computer-readable medium storing a program for causing an inspection apparatus to inspect physiological information of a subject being tested to execute a process, the process comprising:
receiving a signal corresponding to the physiological information;
displaying information corresponding to the signal in real time on a display during the inspection;
storing data corresponding to the information;
generating based on the data a data file for enabling browsing of the information in response to a request from an external apparatus; and
transmitting the data file to the external apparatus.
